(19) United States
(12) Patent Application Publication
(10) Pub. No.: US 2023/0206038 A1
(43) Pub. Date: Jun. 29, 2023

(54) A MODELING METHOD FOR ARTIFICIAL NEURAL PATHWAY ACROSS ENCEPHALIC REGIONS

(71) Applicant: ZHEJIANG UNIVERSITY, HANGZHOU, Zhejiang Province (CN)

(72) Inventors: GANG PAN, HANGZHOU, ZHEJIANG PROVINCE (CN); YIWEN WANG, HANGZHOU, ZHEJIANG PROVINCE (CN); CUNLE QIAN, HANGZHOU, ZHEJIANG PROVINCE (CN)

(21) Appl. No.: 17/927,824

(22) PCT Filed: May 23, 2021
(86) PCT No.: PCT/CN2021/095360

§ 371 Date: Nov. 25, 2022

(30) Foreign Application Priority Data
May 25, 2020 (CN) 202010447142.8
May 21, 2021 (CN) 202110559426.0

(51) Int. Cl. G06N 3/049; G06N 3/10

(52) U.S. Cl. CPC G06N 3/049; G06N 3/105

(57) ABSTRACT

The present invention discloses a prediction method of brain region pulse neural signals, comprising the following steps: 1) synchronously acquiring pulse signals of neural groups in multiple brain regions; 2) calibrating the pulse signals of the neural groups; 3) pre-processing the pulse signals of the neural groups; 4) constructing a non-discrete neural pulse sequence kernel function; 5) performing dimensionality reduction on a reproducing Kernel Hilbert Space; 6) solving for an artificial neural pathway model in the reproducing Kernel Hilbert Space; 7) evaluating the artificial neural pathway model; and 8) visualizing the artificial neural pathway model. The method uses the non-discrete neural pulse sequence kernel function input on the basis of a time sequence neural pulse, has higher output signal prediction accuracy, higher computing efficiency, and higher stability performances, and is used for guiding the rehabilitation of a cognitive nerve function.

### TECHNICAL FIELD

The present invention relates to a field of neural engineering, in particular to a modeling method for artificial neural pathway across brain regions.

### DESCRIPTION OF RELATED ART

There are neural pathway between the main functional regions of the brain, which is composed of neuron synapses. Different brain regions communicate through neural pathways to realize information transmission and have normal cognitive functions (such as understanding, memory, etc.). When encountering brain damage, the neural pathways may be cut off, and cognitive neural function will be reduced or even lost.

However, with the continuous development of micro-electrode array technology and the continuous deepening of modern medicine research on brain functions, people can achieve synchronous collection of neural pulse signals in the two groups of brain regions. Through the analysis of the correlation between the collected signals, people can recognize the functional relationship between various brain regions, so as to further build an artificial neural pathway to supplement or replace the damaged original neural pathway. For example, the patent application with the publication number of CN106529186A discloses a prediction method for pulse neural signals in the brain region, and the patent application with the publication number of CN112101535A discloses a signal processing method and related devices for pulse neurons.

Due to the complexity and nonlinearity of neural communication, the input signal flux is large, and the artificial neural pathway needs to be implanted into the human brain to work. Therefore, the mathematical model of the artificial neural pathway needs to have efficient and stable nonlinear expression ability, which challenges the traditional linear inefficient model.

### SUMMARY OF THE INVENTION

In view of the above, in view of the demand for the rehabilitation of existing cognitive neural function, the object of the present invention is to provide a modeling method of artificial neural pathway across brain regions. On the basis of synchronous recording of neural pulse signals at multiple points, an artificial neural pathway model that can accurately and efficiently predict the neural pulse signals of the output brain region is constructed. The application of the artificial neural pathway model is used to guide the rehabilitation of cognitive neural function.

In order to achieve the object of the present invention, the technical solution provided by the embodiment of the present invention is:
A modeling method of artificial neural pathway across brain regions, comprising the following steps:
Conducting synchronous collection of neural pulse signals for multiple neural groups of an input brain region and an output brain region. According to the waveform characteristics of the neural pulse signals, calibrating the time of releasing neural pulse signals and the corresponding neurons.

Discreting the time slot of all neural pulse signals. Screening and filtering all the neurons in the input brain region and the output brain region based on the neural pulse release rates.

For each remaining output neuron screened and filtered in the output brain region, screening multiple input neurons from the input brain region as sample neurons according to the correlation between the neural pulse signals. Constructing a time sequence input neural pulse history of the sample neurons according to the time of releasing neural pulse signals .

A non-discrete neural pulse sequence kernel function is constructed based on the time sequence input neural pulse history of the input neuron, and the time sequence input neural pulse history is projected into a Reproducing Kernel Hilbert Space. Reducing the dimension of the Reproducing Kernel Hilbert Space by clustering the time sequence input neural pulse history.

In the Reproducing Kernel Hilbert Space of reduced dimension, the non-discrete neural pulse sequence kernel function after linear weighting is used as the predictive value of the neural pulse signal of the output neuron. Optimizing the weight parameters of linear weighting with the objective of maximizing the likelihood function of the predicted value of the neural pulse signal of the output neuron, the linear mapping relationship consisting of the weight parameters constitutes the artificial neural pathway model.

The beneficial effects of the technical solution provided by the above embodiments are at least reflected in:

Based on the neural pulse signals of multiple neurons collected synchronously in multiple regions, the time sequence input neural pulse history is used as the input variable, based on the non-discrete neural pulse sequence kernel function method, an artificial neural pathway model that can predict neural pulse signals in real time is constructed. The model not only accepts the time sequence input neural pulse history input, which improves the storage and calculation efficiency. At the same time, because of the use of kernel function method, the model has a high degree of nonlinear expression ability and the stable solution of the global optimal model, so the model has a stable, efficient and accurate prediction ability of the output neural pulse signal. The neural pulse signal of the output neuron are predicted by the artificial neural pathway model, the neural pulse signal of the input neuron and the neural pulse signal of the output neuron constitute the artificial neural pathway, which can be used to guide the rehabilitation of cognitive neural function.

### BRIEF DESCRIPTION OF THE DRAWINGS

In order to more clearly explain the embodiments of the present invention or the technical solution in the prior art, the following will briefly introduce the drawings needed in the embodiments or the prior technical description. Obviously, the drawings in the following description are only some embodiments of the present invention. For ordinary technical personnel in the art, they can also obtain other drawings based on these drawings without paying creative labor.

FIG. 1 is a flow chart of the modeling method for artificial neural pathway across brain regions provided by an embodiment;

FIG. 2 is a schematic diagram of the artificial neural pathway working in the brain provided by an embodiment.

### DETAILED DESCRIPTION OF THE INVENTION

In order to achieve the object, technical scheme and advantages of the present invention clearer, the present invention is further described in detail below in combination with the drawings and embodiments. It should be understood that the specific embodiments described herein are only used to explain the present invention and do not limit the scope of protection of the present invention.

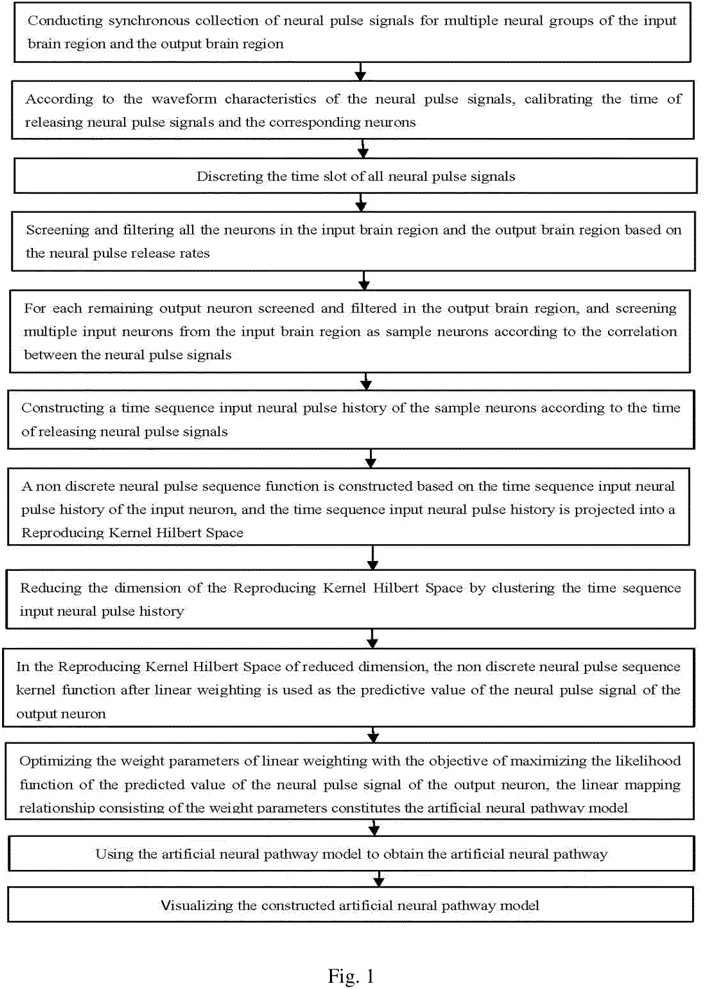

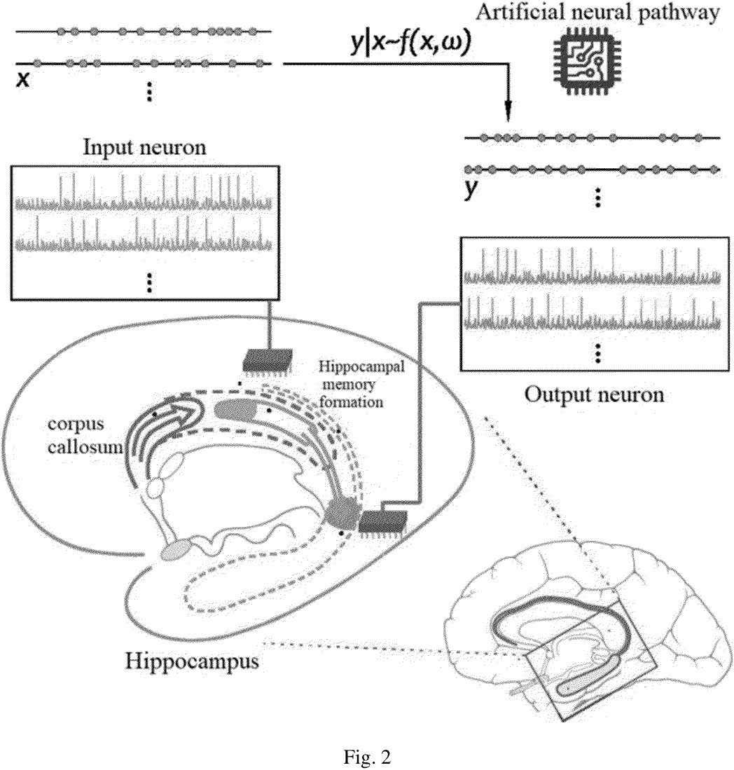

FIG. 1 is a flow chart of the modeling method for artificial neural pathway across brain regions provided by an embodiment. FIG. 2 is a schematic diagram of the artificial neural pathway working in the brain provided by an embodiment. As shown in FIG. 1 and FIG. 2, the modeling method of artificial neural pathway across brain regions provided by the embodiment includes the following steps:

Step 1, conducting synchronous collection of neural pulse signals for multiple neural groups of the input brain region and the output brain region.

In order to establish the artificial neural pathway, the input brain region corresponding to an input signal and the output brain region corresponding to an output signal need to be determined. On the basis of determining the input brain region and the output brain region, the synchronous collection of neural pulse signals of multiple neural groups in the brain regions can be carried out. Specifically, more than two groups of channel electrode arrays can be buried at multiple locations on the surface of the cerebral cortex of the subject (generally the input brain region and the output brain region). When the subject performs relevant tasks, the neural pulse released by neurons in the brain region can be observed and recorded synchronously in real time, realizing the synchronous collection of neural pulse signals.

Step 2, according to the waveform characteristics of the neural pulse signals, calibrating the time of releasing neural pulse signals and the corresponding neurons.

The neural pulse signals released by different neurons are recorded by the same electrode, but the waveform characteristics of releasing pulse are different, the time of releasing neural pulse signals and neurons can be distinguished by analyzing the waveform characteristics. In the embodiment, the waveform characteristics of the neural pulse signals include wave peak value, wave valley value, peak valley time interval, etc., calibrating the time of releasing neural pulse signals and the corresponding neurons according to the waveform characteristics.

Step 3, discreting the time slot of all neural pulse signals.

In the embodiment, discreting the time slot is performed on the neural pulse signals of the output brain region, comprising:

Dividing the collected neural pulse signals according to the fixed time slot width, and recording the time slot of the neural pulse signals in the time slot as 1, otherwise as 0, so as to complete the discreting. For example, the fixed time slot width can be set to 10 milliseconds, and the discreting of the neural pulse signals can be completed through the 10 millisecond time slot width.

Step 4, Screening and filtering all the neurons in the input brain region and the output brain region based on the neural pulse release rates.

Due to the distribution of the release rates of the collected neural pulse signals are distributed widely, and some neural pulse release rates are too high or too low, screening and filtering these neurons and the corresponding neural pulse signals is necessary. In the embodiment, all neurons in the input brain region and the output brain region are screened and filtered according to the neural pulse release rates, comprising:

For all the neurons in the input brain region and output brain region, the neurons that are not within a threshold range of neural pulse firing rate are filtered out according to a set threshold range of neural pulse release rate. In the embodiment, the threshold range of neural pulse release rate can be set as [2 Hz, 40 Hz], and neurons with neural pulse release rate outside the range of [2 Hz, 40 Hz] can be screened out and filtered out.

Step 5, For each remaining output neuron screened and filtered in the output brain region, and screening multiple input neurons from the input brain region as sample neurons according to the correlation between the neural pulse signals.

On the basis of filtering neurons through the threshold range of the neural pulse release rate, further screening of neurons is required to obtain neurons that can establish an artificial neural pathway models as sample data, named as sample neurons. In the embodiment, for each remaining output neuron screened and filtered in the output brain region, multiple input neurons are selected from the input brain region as sample neurons according to the correlation between the neural pulse signals, comprising:

For each remaining output neuron screened and filtered in the output brain region, a mutual information between the output neurons and each input neuron in the input brain region is calculated, and multiple input neurons with the highest mutual information before n are selected as sample neurons.

In the embodiment, n can be taken as 10. After calculating the mutual information value of each channel of pulse neural signals in the input brain region and the output brain region, for each channel of pulse neural signals in the output brain region, the input neurons of the first 10 input brain regions with the highest mutual information value are selected as the sample neurons, the neural pulse signals corresponding to the sample neurons are used as the input data of the artificial neural pathway model to predict the neural pulse signal of the output neuron.

The calculation method of the mutual information is:

$$I \left({x}_{i} , {y}_{j}\right) = {\sum }_{{x}_{t} ∈ {x}_{i}} {\sum }_{{y}_{t} ∈ {y}_{i}} p \left({x}_{t ,} {y}_{t}\right) log \left(\frac{p \left({x}_{t ,} {y}_{t}\right)}{p \left({x}_{t}\right) p \left({y}_{t}\right)}\right)$$

wherein, xᵢ is the neural pulse signal of the ith input neuron in the input brain region, yⱼ is the neural pulse signal of the jth output neuron in the output brain region, p(xₜ, yₜ) is the joint probability of the simultaneous occurrence of the event xₜ and yₜ, p(xₜ) and p(yₜ) respectively represent the probability of the occurrence of the event xₜ and yₜ. Since the pulse neural signals in the original brain region has been discreted, the value range of the event xₜ and yₜ is between {0,1}.

Step 6, constructing a time sequence input neural pulse history of the sample neurons according to the time of releasing neural pulse signals .

On the basis of obtaining the sample neurons, at each discrete time interval, in the relevant input neurons, the latest history input neural pulse time is listed correspondingly to build the time sequence input neural pulse history, that is, the time sequence input neural pulse history is an event sequence composed of the time released by the input neuron neural pulse. In the embodiment, constructing a time sequence input neural pulse history of the sample neurons according to the time of releasing neural pulse signals, comprising:

The time sequence formed by the time of sample neurons releasing neural pulse signals are used as the time sequence input neural pulse history, which is expressed as

$${x}_{k} = {\left\{{τ}_{k}^{m , n}\right\}}_{m \times n} ,$$

where,

$${τ}_{k}^{m , n}$$

represents the m-th releasing neural pulse signal time before tₖ time of the nth sample neuron.

Step 7, a non-discrete neural pulse sequence kernel function is constructed based on the time sequence input neural pulse history of the input neuron, and the time sequence input neural pulse history is projected into a Reproducing Kernel Hilbert Space.

On the basis of obtaining the time sequence input neural pulse history of the input neuron, an inner product function corresponding to two input neural pulse history is used to construct a neural pulse sequence function, and the input signal is projected into the Reproducing Kernel Hilbert Space. In the embodiment, the non-discrete neural pulse sequence kernel function is constructed based on the time sequence input neural pulse history of the input neuron, comprising:

The non-discrete neural pulse sequence kernel function κ(▪) is expressed as:

$$κ \left({χ}_{i} , {χ}_{j}\right) = exp \left(- \frac{d i s t {\left({χ}_{i} , {χ}_{j}\right)}^{2}}{2 {σ}_{R}^{2}}\right)$$

$$d i s t {\left({χ}_{i} , {χ}_{j}\right)}^{2} = {κ}_{c} \left({χ}_{i} , {χ}_{i}\right) - 2 {κ}_{c} \left({χ}_{i} , {χ}_{j}\right) + {κ}_{c} \left({χ}_{j} , {χ}_{j}\right)$$

$${κ}_{c} \left({χ}_{i} , {χ}_{j}\right) = {\sum }_{n = 1}^{N} {\sum }_{m 2 = 1}^{M} {\sum }_{m 1 = 1}^{M} e x p \left\{- \frac{\left[{τ}_{i}^{m 1 , n} - {τ}_{j}^{m 2 , n}\right]}{2 {σ}_{S}^{2}}\right\}$$

wherein, dist(χᵢ,χⱼ) represents the distance between the i-th time sequence input neural pulse history χᵢ and the j-th time sequence input neural pulse history χⱼ, using for measurement the dissimilarity degree of χᵢ and χⱼ, κ_{c}(▪) represents the cross firing intensity kernel function between the two time sequence input neural pulse history, m1 and m2 are indexes of the release number of neural pulse signals, M represents the total release number of neural pulse signals, and N represents the total number of neurons.

Step 8, reducing the dimension of the Reproducing Kernel Hilbert Space by clustering the time sequence input neural pulse history.

In order to limit the linear growth of the dimension of the Reproducing Kernel Hilbert Space with the input sample data and avoid the over fitting problem of the input and output models, reducing the dimension of the Reproducing Kernel Hilbert Space is necessary. In the embodiment, reducing the dimension of the Reproducing Kernel Hilbert Space by clustering the time sequence input neural pulse history, comprising:

According to the distance between the time sequence input neural pulse history, the time sequence input neural pulse history is clustered and the cluster center is determined. The time sequence input neural pulse history corresponding to the cluster center is used to form the Reproducing Kernel Hilbert Space of reduced dimension.

In the embodiment, during dimension reduction processing, the distance dist(χᵢ,χⱼ) between any two input time sequence input neural pulse history, K center point algorithm is used to obtain some drastic results and corresponding cluster center points, and then the cluster center points are used as the feature points representing the Reproducing Kernel Hilbert Space to form the Reproducing Kernel Hilbert Space of reduced dimension.

Step 9, in the Reproducing Kernel Hilbert Space of reduced dimension, the non-discrete neural pulse sequence kernel function after linear weighting is used as the predictive value of the neural pulse signal of the output neuron.

In the embodiment, in the Reproducing Kernel Hilbert Space of reduced dimension, on the basis of the neural pulse sequence kernel function κ(▪) constructed between the time sequence input neural pulse history corresponding to each cluster center, the prediction value of the neural pulse signal of the output neuron based on the time sequence input neural pulse history is obtained by linear weighting.

Step 10, optimizing the weight parameters of linear weighting with the objective of maximizing the likelihood function of the predicted value of the neural pulse signal of the output neuron, the linear mapping relationship consisting of the weight parameters constitutes the artificial neural pathway model.

In the embodiment, the neural pulse sequence kernel function constructed by two time sequence input neural pulse history is used as a variable, and the weighted linear mapping relationship of the variable constitutes the artificial neural pathway model. When solving the output neural pulse prediction model, the maximum likelihood estimation framework is used, and the iterative reweighted least squares method is used to obtain the global optimal solution of the neural pathway model weight. Specifically, optimizing the weight parameters of linear weighting with the objective of maximizing the likelihood function of the predicted value of the neural pulse signal of the output neuron, by constantly changing the weight parameters, the likelihood function of the neural pulse signal of the output neuron is maximized until it reaches the global optimal value, so as to obtain the artificial neural pathway model.

Step 11, using the artificial neural pathway model to obtain the artificial neural pathway.

In the embodiment, when the artificial neural pathway model is used to obtain the artificial neural pathway, the neural pulse signals input into the brain region are collected for discretization, and screened and filtered, the time sequence input neural pulse history of the input neurons is constructed. Based on the time sequence input neural pulse history constructed, the neural pulse signals of the output neurons are predicted using the artificial neural pathway model, an artificial neural pathway is formed according to input neurons and output neurons.

Step 12: Visualizing the constructed artificial neural pathway model.

In the embodiment, the constructed artificial neural pathway model is also visualized. The process is as follows: the neural pulse signal contained in the time sequence neural pulse history corresponding to the cluster center are taken as the representative neural pulse signal of the input neuron. After smoothing the neural pulse signal, combining the corresponding relationship between the pulse neural signal of input neurons obtained by combining the corresponding weight parameters and the pulse neural signal of output neurons. After smoothing any two neural pulse signals in the neural pulse signals of multiple input neurons corresponding to multiple cluster centers, the corresponding weight parameters are combined to show the interaction of the pulse neural signals of two input neurons in determining the neural pulse signals of output neurons.

In the embodiment, the artificial neural pathway model is also evaluated. The discrete time reproducing Kolmanov Smirnov statistics is used as the evaluation standard to measure the effect of the model, and the goodness of fit of the pulse neural signal of the output neuron predicted by the artificial neural pathway model is calculated. The specific calculation method of discrete time reproducing Kolmogorov Smirnov statistics is as follows:
(1) In order to eliminate the impact of discrete time sampling, a new sequence is generated based on the predicted release probability of output neurons. The specific form is:
$$q \left(t\right) = - log \left(1 - λ \left(t\right) Δ\right)$$
(2) The sequence q(t) is cut according to the pulse sequence generated by the neuron to be predicted, and then the q(t) value between two adjacent pulse signals is integrated, and the deviation generated by discrete time processing is corrected. The specific form is as follows:
$$ζ \left(i\right) = {\sum }_{t = {t}_{i} + 1}^{{t}_{i + 1}} q \left(t\right) + q \left({t}_{i + 1}\right) \frac{δ \left(i\right)}{Δ}$$
wherein, tᵢ refers to the occurrence time of the ith pulse signal of the neuron to be predicted, tᵢ₊₁ refers to the occurrence time of the i+1th pulse signal of the neuron to be predicted, and δ(i) is a random variable acquired in the following form:
$$δ \left(i\right) = - \frac{Δ}{q \left({t}_{i + 1}\right)} log \left(1 - r \left(i\right)\right) \left(\left(1 - {e}^{- q \left({t}_{i + 1}\right)}\right)\right)$$
wherein, r(i) is a random variable uniformly distributed between [0,1].
(3) Scaling up and down ζ(i) to obtain:
$$z \left(i\right) = 1 - {e}^{- {ζ}^{\left(i\right)}}$$
According to the Kolmogorov-Smirnov test theory, the distribution of set { z(i) } should follow the uniform distribution between [0,1];
(4) The set { z(i) } is rearranged according to the value from small to large, and compared with the uniform distribution between the standard [0,1] drawn in the coordinate system at the same time. The horizontal axis is set { z(i) }, and the vertical axis is the standard uniform distribution. Calculating the ratio between the effect measurement value and the range of 95% confidence interval, the maximum difference between the two values on the vertical axis is taken as the effect measurement value, also known as the DTR-KS measurement value.

The modeling method of the artificial neural pathway across the brain region provided above can accept non-discrete neural pulse release time input, and the input mode can efficiently store the neural pulse signal. The modeling method has good nonlinear expression ability and better output prediction accuracy. The model has a global optimal solution, so the solution of the model is stable, and the prediction results and performance of the model are relatively stable.

### Experimental Example

The experiment was carried out on the neural pulse signal of the monkey’s PMd brain region and M1 brain region, the background of the signal was acquired when the monkey was performing a four-way center out task, the effective duration of the signal was 636.4 seconds, including 127 channels of PMd brain region and 102 channels of M1 brain region. After screening, 100 channels of effective PMd signal pathway and 54 channels of effective M1 signal pathway were obtained.

The experimental results are compared with those obtained from other pulse signal models, including neural network model (NN), second-order Laguerre Volterra model (LVM), generalized linear model (GLM), etc. Table 1 shows the horizontal comparison of the prediction effect of this method and other methods on pulse signals. The DTR-KS statistic is the abbreviation of discrete time rescaling Kolmogorov Smirnov test(Discrete Time Rescaling Kolmogorov Smirnov test). If the value is less than 1, the output prediction will pass the discrete time rescaling Kolmogorov Smirnov test. The closer the value is to 0, the better the prediction effect of this model will be.

TABLE 1

Effect comparison between this method and other prediction methods

| Prediction method | DTR-KS statistic | The number of M1 brain region signal channels with the best effect obtained by this method |
|---|---|---|
| NN | 1.22 ± 0.38 | 21 |
| LVM | 1.36 ± 0.55 | 18 |
| GLM | 1.39 ± 0.57 | 16 |
| this method | 1.20 ± 0.37 | 28 |

The above specific embodiments have described the technical scheme and beneficial effects of the present invention in detail. It should be understood that the above is only the best selected embodiment of the present invention, and is not used to limit the present invention. Any modification, supplement and equivalent replacement made within the principle scope of the present invention should be included in the protection scope of the present invention.

1. A modeling method of artificial neural pathway across brain regions, comprising the following steps:
conducting synchronous collection of neural pulse signals for multiple neural groups of an input brain region and an output brain region; according to the waveform characteristics of the neural pulse signals, calibrating the time of releasing neural pulse signals and the corresponding neurons;
discreting the time slot of all neural pulse signals; screening and filtering all the neurons in the input brain region and the output brain region based on the neural pulse release rates;
for each remaining output neuron screened and filtered in the output brain region, screening multiple input neurons from the input brain region as sample neurons according to the correlation between the neural pulse signals; constructing a time sequence input neural pulse history of the sample neurons according to the time of releasing neural pulse signals;
constructing a non-discrete neural pulse sequence kernel function based on the time sequence input neural pulse history of the input neuron, and projecting the time sequence input neural pulse history into a Reproducing Kernel Hilbert Space; reducing the dimension of the Reproducing Kernel Hilbert Space by clustering the time sequence input neural pulse history;
in the Reproducing Kernel Hilbert Space of reduced dimension, using the non-discrete neural pulse sequence kernel function after linear weighting as the predictive value of the neural pulse signal of the output neuron; optimizing the weight parameters of linear weighting with the objective of maximizing the likelihood function of the predicted value of the neural pulse signal of the output neuron, constituting the linear mapping relationship consisting of the weight parameters the artificial neural pathway model.

2. The modeling method of artificial neural pathway across brain regions according to claim 1, wherein, the waveform characteristics of the neural pulse signals include wave peak value, wave valley value, and peak valley time interval, calibrating the time of releasing neural pulse signals and the corresponding neurons according to the waveform characteristics.

3. The modeling method of artificial neural pathway across brain regions according to claim 1, wherein, discreting the time slot is performed on the neural pulse signals of the output brain region, comprising: dividing the collected neural pulse signals according to the fixed time slot width, and recording the time slot of the neural pulse signals in the time slot as 1, otherwise as 0, so as to complete the discreting.

4. The modeling method of artificial neural pathway across brain regions according to claim 1, wherein, all neurons in the input brain region and the output brain region are filtered according to the neural pulse release rates, comprising: for all the neurons in the input brain region and output brain region, the neurons that are not within a threshold range of neural pulse firing rate are filtered out according to a set threshold range of neural pulse release rate.

5. The modeling method of artificial neural pathway across brain regions according to claim 1, wherein, for each remaining output neuron screened and filtered in the output brain region, multiple input neurons are selected from the input brain region as sample neurons according to the correlation between the neural pulse signals, comprising:
for each remaining output neuron screened and filtered in the output brain region, a mutual information between the output neurons and each input neuron in the input brain region is calculated, and multiple input neurons with the highest mutual information before n are selected as sample neurons.

6. The modeling method of artificial neural pathway across brain regions according to claim 1, wherein, constructing a time sequence input neural pulse history of the sample neurons according to the time of releasing neural pulse signals, comprising:
the time sequence formed by the time of sample neurons releasing neural pulse signals are used as the time sequence input neural pulse history, which is expressed as
$${x}_{k} = {\left\{{τ}_{k}^{m , n}\right\}}_{m \times n} ,$$
where,
$${τ}_{k}^{m , n}$$
represents the m-th releasing neural pulse signal time before tₖ time of the nth sample neuron.

7. The modeling method of artificial neural pathway across brain regions according to claim 1, wherein, the non-discrete neural pulse sequence kernel function is constructed based on the time sequence input neural pulse history of the input neuron, comprising:
the non-discrete neural pulse sequence kernel function κ(▪) is expressed as:
$$κ \left({χ}_{i} , {χ}_{j}\right) = exp \left(- \frac{d i s t {\left({χ}_{i} , {χ}_{j}\right)}^{2}}{2 {σ}_{R}^{2}}\right)$$
$$d i s t {\left({χ}_{i} , {χ}_{j}\right)}^{2} = {κ}_{c} \left({χ}_{i} , {χ}_{i}\right) - 2 {κ}_{c} \left({χ}_{i} , {χ}_{j}\right) + {κ}_{c} \left({χ}_{j} , {χ}_{j}\right)$$
$${κ}_{c} \left({χ}_{i} , {χ}_{j}\right) = {\sum }_{n = 1}^{N} {\sum }_{m 2 = 1}^{M} {\sum }_{m 1 = 1}^{M} e x p \left\{- \frac{{\left[{τ}_{i}^{m 1 , n} - {τ}_{j}^{m 2 , n}\right]}^{2}}{2 {σ}_{S}^{2}}\right\}$$
wherein, dist(χᵢ,χⱼ) represents the diatance between the ith time sequence input neural pulse history χᵢ and the jth time sequence input neural pulse history χⱼ, using for measurement the dissimilarity degree of χᵢ and χⱼ, κ_{c}(▪) represents the cross firing intensity kernel function between the two time sequence input neural pulse history.

8. The modeling method of artificial neural pathway across brain regions according to claim 1, wherein, reducing the dimension of the Reproducing Kernel Hilbert Space by clustering the time sequence input neural pulse history, comprising:
according to the distance between the time sequence input neural pulse history, the time sequence input neural pulse history is clustered and the cluster center is determined. The time sequence input neural pulse history corresponding to the cluster center is used to form the Reproducing Kernel Hilbert Space of reduced dimension.

9. The modeling method of artificial neural pathway across brain regions according to claim 1, wherein, also comprising: the neural pulse signals input into the brain region are collected for discretization, and screened and filtered, the time sequence input neural pulse history of the input neurons is constructed; based on the time sequence input neural pulse history constructed, the neural pulse signals of the output neurons are predicted using the artificial neural pathway model, an artificial neural pathway is formed according to input neurons and output neurons.

10. The modeling method of artificial neural pathway across brain regions according to claim 1, wherein, also comprising: the constructed artificial neural pathway model is also visualized, the process is as follows: the neural pulse signal contained in the time sequence neural pulse history corresponding to the cluster center are taken as the representative neural pulse signal of the input neuron; after smoothing the neural pulse signal, combining the corresponding relationship between the pulse neural signal of input neurons obtained by combining the corresponding weight parameters and the pulse neural signal of output neurons; after smoothing any two neural pulse signals in the neural pulse signals of multiple input neurons corresponding to multiple cluster centers, the corresponding weight parameters are combined to show the interaction of the pulse neural signals of two input neurons in determining the neural pulse signals of output neurons.
